(19) United States
(12) Patent Application Publication
(10) Pub. No.: US 2022/0401655 A1
(43) Pub. Date: Dec. 22, 2022

(54) PLUNGER ROD AND SYRINGE ADAPTED FOR COMPRESSION FAILURE

(71) Applicant: KRUI Medical AB, Helsingborg (SE)

(72) Inventor: Per KNUTSSON, Helsingborg (SE)

(73) Assignee: KRUI Medical AB, Helsingborg (SE)

(21) Appl. No.: 17/773,713

(22) PCT Filed: Nov. 5, 2020
(86) PCT No.: PCT/SE2020/051062

§ 371 Date: May 2, 2022

(30) Foreign Application Priority Data
Nov. 6, 2019 (SE) 1951268-0

(51) Int. Cl. A61M 5/315; A61M 5/50

(52) U.S. Cl. CPC A61M 5/31515; A61M 5/50; A61M 2005/5073

(57) ABSTRACT

A plunger rod (104) for use in connection with a syringe barrel (102) is provided. The plunger rod (104) comprises a distal plunger top (105), having a proximal bottom surface (113), and a proximal elongated shaft (106) comprising a proximal end (107a) adapted to receive an applied force along an axis (1000) and a distal end plate (107b) adapted to transmit said applied force along said axis (1000). The plunger rod (104) is adapted to be inserted into the barrel (102) of a syringe (100) extending along the axis (1000). The distal plunger top (105) and the proximal elongated shaft (106) are connected to each other by a releasable connection (115). The releasable connection (115) comprises: distal top rupture means (116) comprising at least one distal top rib (125a) disposed eccentrically from the axis (1000) on the proximal bottom surface (113), and proximal shaft rupture means (117) comprising at least one proximal shaft rib (125b) disposed eccentrically from the axis (1000) on the distal end plate (107b). Each said at least one distal top rib (125a) is connected to a corresponding at least one proximal shaft rib (125b), said ribs (125a, 125b) being connected at a contact area (118), and wherein said distal top rupture means (116) is configured to detach from the proximal shaft rupture means (117) due to compression failure when the distal plunger top (105) is pushed into a distal end (110) of the barrel (102). A syringe is also provided.

### TECHNICAL FIELD

The present invention relates to a plunger rod of a syringe for preventing re-usage of the syringe which can be manufactured at a cost similar to a conventional syringe. The present invention further relates to a syringe comprising said plunger rod.

### BACKGROUND

Injections are among the most common health care procedures, with at least 16 billion injections administered globally each year. Multiple use of the same syringe to perform injections to more than one person causes the spread of several infectious diseases. It is estimated that over 2 million patients and users each year are infected with severe blood transmittable diseases, such as HIV, Hepatitis B and C due to the misuse of syringes. A majority of these injections are performed using conventional syringes or pre-filled conventional syringes available on the market.

In 2015, the World Health Organisation (WHO) set a time limit that all syringes worldwide should be exchanged for so called “smart syringes”, such as auto-disabled (AD) syringes, by 2020.

The AD syringes available on the market suffer from several drawbacks. Most smart syringes are based on locking mechanisms between the plunger rod and the syringe barrel. When the plunger rod is inserted into and reaches the front part of the cylinder barrel of the syringe, the locking mechanism is activated. During an attempt to retract the plunger rod, a tensile failure occurs and the plunger rod is broken into two separate parts. Thus, reuse of the syringe is prevented. Such AD syringes are for example described in US 2013/0110044 A1.

However, these types of syringes have a complex design. For instance, the inside wall of the barrel may comprise a flange and the piston rod is correspondingly designed to engage the flange. Such difficult designs result in production costs substantially higher than those of conventional syringes.

Another type of AD syringe is a syringe with a sharps protection feature (SIP) and with a reuse Prevention Feature (RUP). The SIP+ RUP syringes are available as manual or automatic versions. In manual versions, the user manually activates the safety features. The automatic retractable models are essentially similar to the manual version, but they include a device, such as a spring, that automatically pulls the needle into the plunger once the plunger rod hits the bottom of the barrel. When pressure is applied to the plunger rod, the spring mechanism in the syringe is activated forcing the needle to be retracted into the syringe barrel. Hence, after one usage, additional injections are made impossible. The risk for injuries due to contact with the needle in for example the disposal process is more or less eliminated. However, this design comprises many different components and a plurality of mounting steps. This in turn yields very high production costs.

Hence, there is a need for an auto-disabled syringe which addresses the disadvantages and shortcomings of the prior art, and which can be manufactured in a cost-efficient manner.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by proposing a solution according to the appended independent claims. Advantageous embodiments are defined in the appended dependent claims.

In a first aspect, there is provided a plunger rod for use in connection with a syringe barrel. The plunger rod comprises a distal plunger top, having a proximal bottom surface, and a proximal elongated shaft which comprises a proximal end adapted to receive an applied force along an axis and a distal end plate adapted to transmit said applied force along said axis. The plunger rod is adapted to be inserted into the barrel of a syringe extending along the axis. The distal plunger top and the proximal elongated shaft are connected to each other by a releasable connection which comprises distal top rupture means having at least one distal top rib disposed eccentrically from the axis on the proximal bottom surface, proximal shaft rupture means having at least one proximal shaft rib disposed eccentrically from the axis on the distal end plate. Each at least one distal top rib is connected to a corresponding at least one proximal shaft rib and the ribs are connected at a contact area. The distal top rupture means is configured to detach from the proximal shaft rupture means due to compression failure when the distal plunger top is pushed into a distal end of the barrel.

In a second aspect, there is provided a syringe comprising a barrel having a syringe tip arranged at a distal end of the barrel. The barrel is adapted to contain a drug, and has a tubular wall extending proximally from said distal end, whereby the barrel extends along an axis. The barrel comprises an aperture arranged at a proximal end of the barrel and the syringe further comprises a plunger rod extending along the axis.

Further advantages will be apparent from the detailed description as well as the appended dependent claims.

### BRIEF DESCRIPTION OF THE DRAWINGS

These and other aspects, features and advantages of which the invention is capable, will be apparent and elucidated from the following description of embodiments of the present invention, reference being made to the accompanying drawings, in which

FIG. 1A is a longitudinal view of a syringe and an isometric view of a plunger rod according to one example;

FIG. 1B is longitudinal views of the syringe and plunger rod according to the example in FIG. 1A;

FIG. 2 is longitudinal views of a syringe and a plunger rod, and an isometric view of said plunger rod according to a second example;

FIG. 3 is longitudinal views of a syringe and a plunger rod, and an isometric view of said plunger rod according to a third example;

FIG. 4 is longitudinal views of a syringe and a plunger rod, and an isometric view of said plunger rod according to a fourth example;

FIG. 5 shows cross-sectional views of a syringe and a plunger rod, and an isometric view of said plunger rod according to a fifth example;

FIG. 6 shows an alternative example of the syringe and the plunger rod shown in FIG. 4 according to a sixth example;

FIG. 7 shows yet another alternative example of the syringe and the plunger rod shown in FIG. 4 according to a seventh example; and

FIG. 8 is longitudinal and isometric views of a pre-filled syringe and a plunger rod according to an eighth example.

### DETAILED DESCRIPTION OF THE EMBODIMENTS

Embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

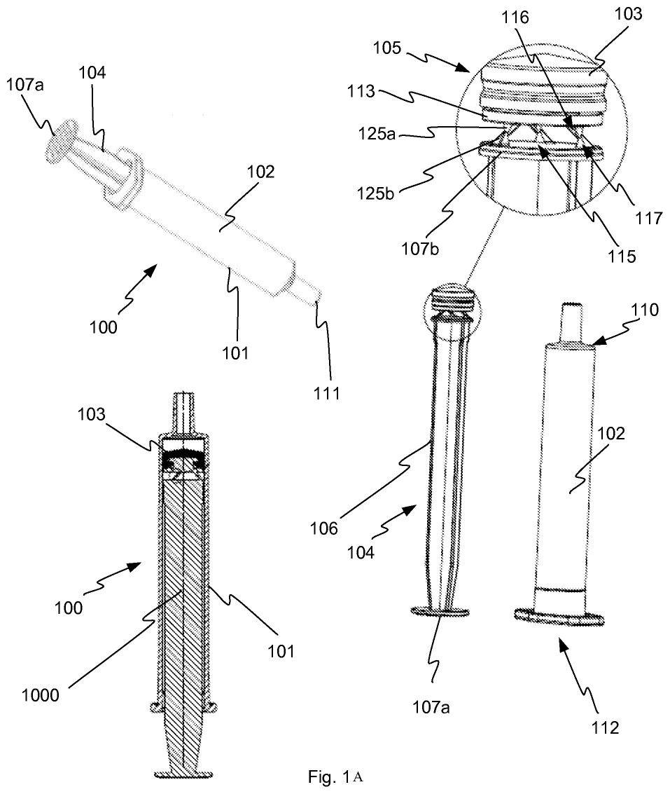

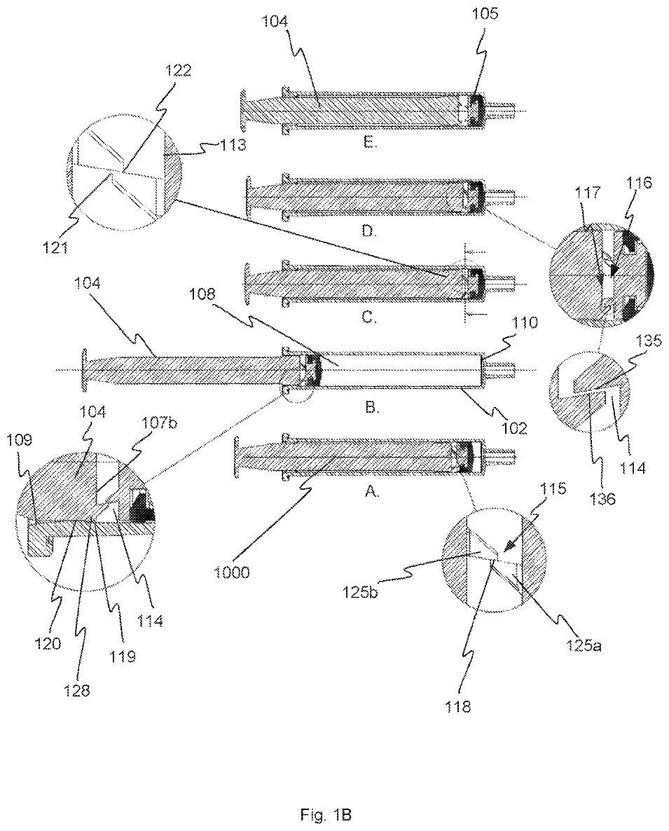

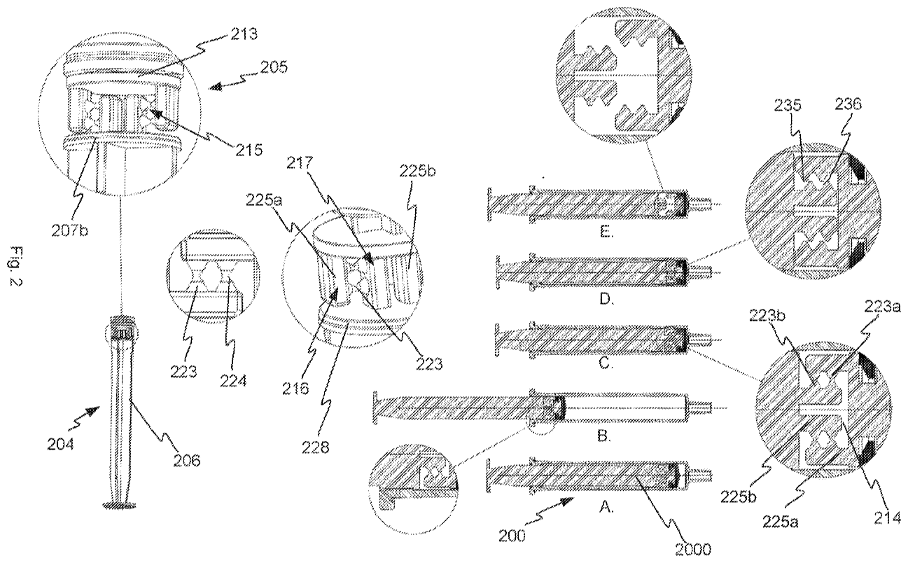

Referring to FIG. 1, one example of a syringe 100 and plunger rod 104 according to the teaching herein is shown. The syringe 100 comprises a barrel 102 having a distal end 110 and a proximal end 112. The distal end 110 comprises a distal tip 111, to which a needle may be attached. The barrel 102 of the syringe 100 may be made of any material suitable for containing a drug, but is preferably made of plastic. The plastic is preferably transparent. The barrel 102 is configured to contain a drug. The drug may be in liquid form to be injected into a patient or user. Further, the barrel 102 has tubular wall 101.

The syringe 100 extends along an axis 1000, which defines the direction in which the barrel 102 extends, e.g. the barrel 102 extends along the axis 1000.

The syringe 100 in FIG. 1 further comprises a plunger rod 104 disposed within the barrel 102. The plunger rod 104 extends along the tubular wall 101 and the axis 1000. A washer 103 is disposed on a distal plunger top 105 of the plunger rod 104. The distal plunger top 105 comprises a proximal bottom plate 113. The plunger rod 104 further comprises an elongate proximal shaft 106 having a proximal end 107a and a distal end plate 107b.

The washer 103 is adapted to be in fluid-tight engagement with the tubular wall 101 of the barrel 102. By being in fluid-tight engagement with the tubular wall 101 of the barrel 102, the drug or liquid contained within the barrel 102 is prevented from leaking out of the syringe 100 in any other direction than through the distal tip 111 (or a needle attached to said distal tip) at the distal end 110 of the barrel 102.

The distal plunger top 105 and the elongate proximal shaft 106 are connected to each other by a releasable connection 115. The releasable connection 115 in FIG. 1A comprises distal top rupture means 116, and proximal shaft rupture means 117. The distal top rupture means 116 are arranged on the proximal bottom surface 113 of the distal plunger top 105. The proximal shaft rupture means 117 are arranged on the distal end plate 107 of the elongate proximal shaft 106. The distal top rupture means 116 on the bottom surface 113 comprises three distal top ribs 125a. The proximal shaft rupture means 117 on the distal end plate 107b comprises three proximal shaft ribs 125b. The distal top ribs 125a are arranged centrally in relation to the proximal shaft ribs 125b. Hence, the proximal shaft ribs 125b are arranged laterally relative the distal top ribs 125a. The connection between the distal top ribs 125a and the proximal shaft ribs 125b will be further explained with reference to FIG. 1B.

Each example in FIGS. 1A-8 shows a different non-limiting embodiment of a releasable connection 115 connecting the distal end top 105 to the proximal elongate shaft 106 of the plunger rod 104. In all embodiments, the axis 1000 defines a longitudinal direction of the syringes and the plunger rods, having a distal end and a proximal end. The axis 1000 extends along a centre of the syringe. The perimeter of the plunger rod and the syringe barrel defines a lateral perimeter.

The distal top ribs 125a and the proximal shaft ribs 125b of the releasable connections 115 disclosed herein are arranged eccentrically in relation to each other and the axis 1000. A rupture of the releasable connections 115 occurs when the plunger rod 104 receives a force applied along the axis 1000, pushing the distal plunger top 105 into the distal end 110 of the barrel 102. When the distal plunger top 105 reaches and is pressed further towards the distal end 110, a compression failure occurs between the distal top rupture means 116 and proximal shaft rupture means 117, which disengages from each other. Thus, the distal plunger top 105 will remain in the barrel 102 when a user extracts the plunger rod 104, and only the elongate proximal shaft 106 is extracted from the barrel 102. Obviously, the syringe 100 cannot be re-used.

FIG. 1B shows cross-sections of use positions A-E of the plunger rod 104 disclosed in FIG. 1A.

Position A (mounting position): the plunger rod 104 is arranged within the barrel 102. The releasable connection 115 connects the distal plunger top 105 to the plunger rod 104.

In this embodiment, a releasable connection 115 is shown in an enlarged view of Position A. The distal end plate 107b and the proximal bottom surface 113 each comprises three separately arranged proximal shaft 125b, and three separately arranged distal top 125a, respectively. However, due to the longitudinal cross sectional views in FIG. 1B, only two distal top ribs 125a and two proximal shaft ribs 125b are visible. Each distal top rib 125a arranged on the bottom surface 113 is paired and connected to a corresponding proximal shaft rib 125b arranged on the distal end plate 107b. A distal top rib 125a is connected to a proximal shaft rib 125b in a contact area 118. Longitudinal cross sections of the distal top ribs 125a and the proximal shaft ribs 125b have a cone like shape or a pyramid like shape.

The distal top ribs 125a of the distal top rupture means 116 and the proximal shaft ribs 125b of the proximal shaft rupture means 117 are connected to each other along the extension of the axis 1000. Each distal top rib 125a is connected to a corresponding proximal shaft rib 125b. The two connected distal top rib 125a and proximal shaft rib 125b are arranged eccentrically relative each other and the axis 1000. A centrally facing surface of one proximal shaft rib 125b (facing towards the axis 1000) are connected with a laterally facing surface of a distal top rib 125a (facing away from the axis 1000) at the contact area 118. Hence, a normal of the surface of the distal top rib 125a will point laterally, out from the centre. A normal of the surface of the proximal shaft rib 125b will point centrally, towards the centre axis 1000. This provides great stability in a lateral/central direction (perpendicular to the axis 1000). The distal top rupture means 116 are arranged centrally, closer to the axis 1000, relative the proximal shaft rupture means 117.

Position B (extracted position): the plunger rod 104 has been extracted, exposing a lumen 108 in the barrel 102. In the enlarged view of position B, the releasable connections 115 are intact and connect the distal plunger top 105 to the elongate shaft 106. Further, the barrel 102 comprises a flattened angle 120, extending from an aperture 109 of the barrel 102 to a circumferential flange 119 formed on an inner wall of the barrel 102. This contributes to that the washer 103 can establish an earlier contact with the inner wall of the barrel 102 during mounting. Further, this design prevents build-up of friction when the washer 103 are to pass the circumferential flange 119. Further, the proximal bottom plate 113 has an angled side edge 128 which also decreases friction when the bottom plate 113 is to pass the circumferential flange 119.

The proximal bottom plate 113 has a diameter smaller than the diameter of the circumferential flange 119 to avoid contact there between, which may damage the releasable connections 115 during mounting. The distal end plate 107b ensures that the piston rod 104 cannot be extracted from the barrel 102 after extraction of a medicament into the lumen 108.

Position C (front position—no pressure load): the plunger rod 104 has been pushed to the distal end 110 of the barrel 102. The pressure load which has been applied has been sufficient to eject the content of the lumen 108 from the syringe 100, but not enough force has been applied to enable a compression failure of the releasable connection 115. The releasable connection 115 is thus intact. A distal interface between the distal top ribs 125a of distal top rupture means 116 and the proximal shaft ribs 125b of the proximal shaft rupture means 117 comprises a weakening in the form of a sharp edge 122. A proximal interface between the distal top rupture means 116 and the proximal shaft rupture means 117 comprises a reinforcement, in the form of a bend 121. The sharp edge 122 act as a weakening of releasable connections 115, accelerating and facilitating the compression failure when the plunger rod 104 is pushed distally as described further with reference to position D. Oppositely, the bend 121 prevents unwanted tension failure during extraction of the plunger rod 104 as shown in position B.

Position D (front position—applied pressure load): the plunger rod 104 has received further force along the axis 1000, and has thus been pressed further towards and into the distal end 110 of the barrel 102. This causes activation and rupture of the releasable connection 115. A compression failure has occurred and the distal top ribs 125a, and the proximal shaft ribs 125b are disconnected from each other. A cavity 114 allows the proximal shaft ribs 125b to be pushed forward and compression failure to occur, even though the distal plunger top 105 has reached and is in contact with the distal end 110. There is no longer any direct contact between the distal top ribs 125a and the proximal shaft ribs 125b, as shown in the enlarged portion of position D. When rupture has occurred, the distal rib 125a has a distal rib fracture surface 135 and the proximal shaft rib 125b has a proximal shaft rib facture surface 136. The distal rib fracture surface 135 points centrally. The proximal shaft rib facture surface 136 points laterally.

Position E (distal plunger top detachment): the distal plunger top 105 is detached from the plunger rod 104 when the user withdraws the plunger rod 104 in the barrel 102. Hence, the syringe 100 cannot be reused. Each distal top rib 125a and proximal shaft rib 125b are angled towards each other (shown in the enlarged view of position A and D). An angle between the centrally facing surface of the rib 125b and the distal end plate 107b is less than 90 degrees. An angle between the laterally facing surface of the rib 125a and the proximal bottom surface 113 is larger than 90 degrees. This decreases the risk of the distal top rupture means 116 and the proximal shaft rupture means 117 engaging each other when the distal plunger top 105 is to be released from the plunger rod 104.

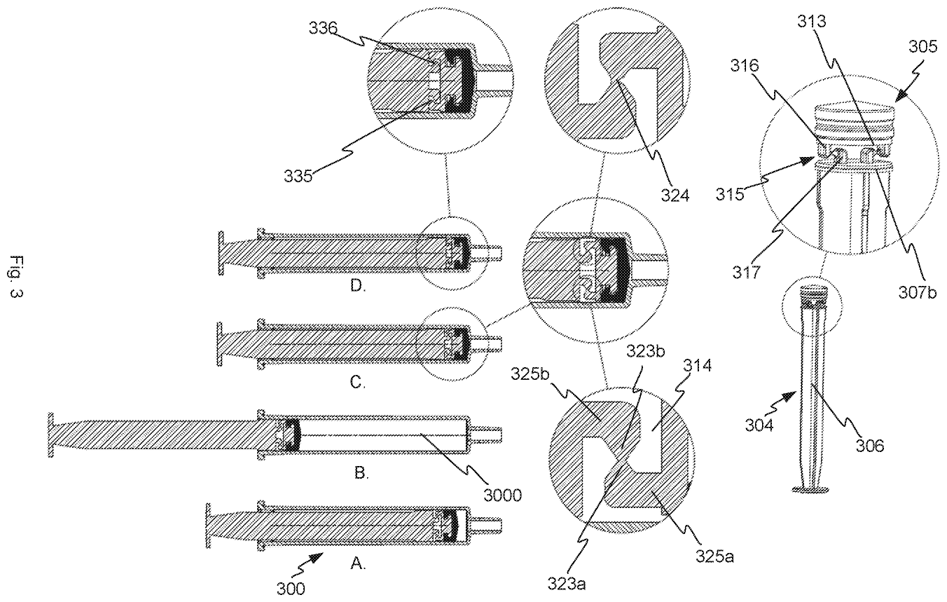

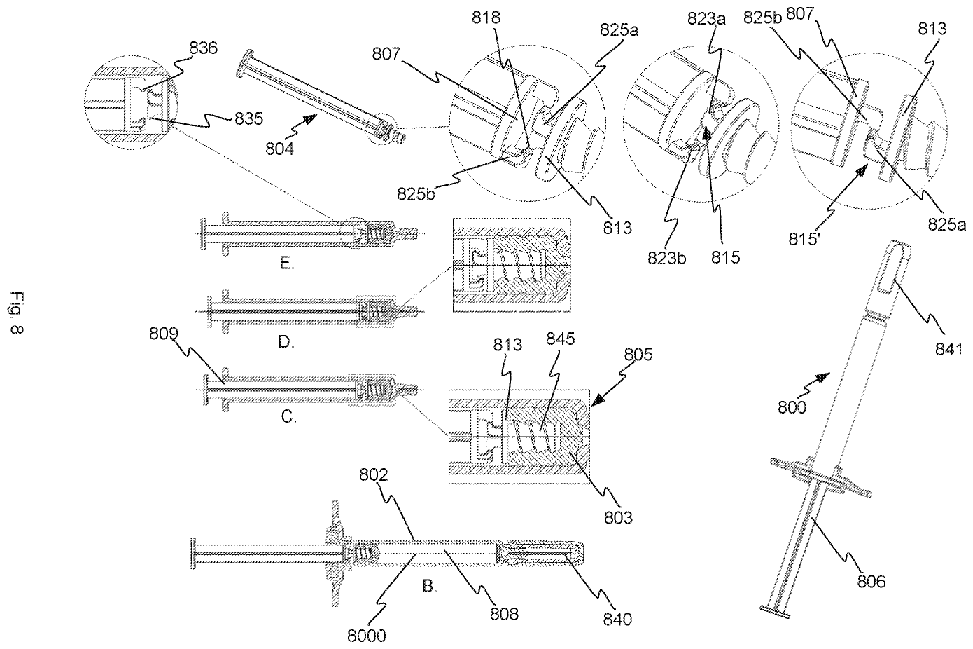

With reference to FIGS. 2-8, additional embodiments of seven plunger rods 204, 304, 404, 504, 604, 704, 804 will be illustrated. The use of the plunger rods in the embodiments shown in FIGS. 2-7 all comprise the positions A-E. FIG. 8 does not comprise Position A, since the example in FIG. 8 relate to a pre-filled AD-syringe 800. Position A is a mounting position. Position B is the extraction position, where the lumen 108 of the barrel 102 is filled with a drug. Position C is the front position where the content of the lumen 108 has been emptied, but no excess force has been applied to the plunger rod 104. Hence, the releasable connection 115, 215, 315, 415, 515, 615, 715, 815 is still intact. Position D is the front position where pressure load has been applied and a compression failure of the releasable connection has taken place. In position E, which is the detachment position, the distal plunger top 105 is released from the plunger rod 104.

FIG. 2 shows a plunger rod 204 according to a second embodiment, comprising a releasable connection 215. The releasable connection 215 comprises distal top rupture means 216 arranged on the proximal bottom surface 213 of the distal end top 205. The proximal shaft rupture means 217 are arranged on the distal end plate 207 of the elongated proximal end shaft 206.

The distal top rupture means 216 and the proximal shaft rupture means 217 each comprises two eccentrically placed distal top ribs 225a and proximal shaft proximal shaft ribs 225b, respectively. The distal top rupture means 216 and the proximal shaft rupture means 217 may comprise more than two eccentrically placed distal top ribs 225a and the proximal shaft ribs 225b. The distal top ribs 225a and the proximal shaft proximal shaft ribs 225b are disposed linearly along a diameter of the bottom surface 213 and the distal end plate 207, respectively. The proximal shaft ribs 225b are positioned centrally, closer to the axis 2000, relative the distal top ribs 225a.

The distal top ribs 225a each comprises two distal rib protrusions 223a and the proximal shaft ribs 225b each comprises two proximal rib protrusions 223b. The distal top ribs 125a and the proximal shaft ribs 125b are connected to each other through the distal rib protrusions 223a and the proximal shaft protrusions 223b. The protrusions are herein also referred to as rupture cones. The distal rib protrusions 223a and the proximal shaft protrusions 223b extend radially from the distal top ribs 225a and the proximal shaft ribs 225b.

The distal rib protrusions 223a on the ribs 225a points centrally. The proximal shaft protrusions 223b points laterally, along the same diameter on which the distal top ribs 225a and proximal shaft ribs 225b are linearly arranged. Each distal rib protrusion 223a is connected to a corresponding proximal rib protrusion 223b. The corresponding distal rib protrusions 223a and proximal rib protrusion 223b are arranged in longitudinal level with each other such that they face each other. The connected distal rib protrusion 223a and proximal rib protrusion 223b form an hour glass shaped connection between the distal top ribs 225a and the proximal shaft ribs 225b.

The hour glass shaped connections formed by the distal rib protrusion 223a and proximal rib protrusion 223b may comprise a weakening groove 224. In FIG. 2, the groove 224 is positioned at the node of the hour glass shaped connection. The groove 224 will accelerate the rupture during compression failure and will enable a sharp/distinct rupture.

The distal rib protrusion 223a and proximal rib protrusion 223b may have different diameters and lengths. A smaller distance between the distal top ribs 225a and the proximal shaft ribs 225b prevents viscoelastic deformation and/or shearing of the distal rib protrusion 223a and proximal rib protrusion 223b before the compression failure occurs, and thus a more effective/distinct compression failure of the distal rib protrusion 223a and proximal rib protrusion 223b. The distal rib protrusion 223a and proximal rib protrusion 223b are linearly positioned on the distal top ribs 225a and the proximal shaft ribs 225b along the axis 2000 making it possible to use more than two distal rib protrusion 223a and proximal rib protrusion 223b.

When rupture has occurred as shown in Position D, the distal rib 225a has a distal rib fracture surface 235 and the proximal shaft rib 225b has a proximal shaft rib facture surface 236. The distal rib fracture surface 235 points centrally. The proximal shaft rib facture surface 236 points laterally.

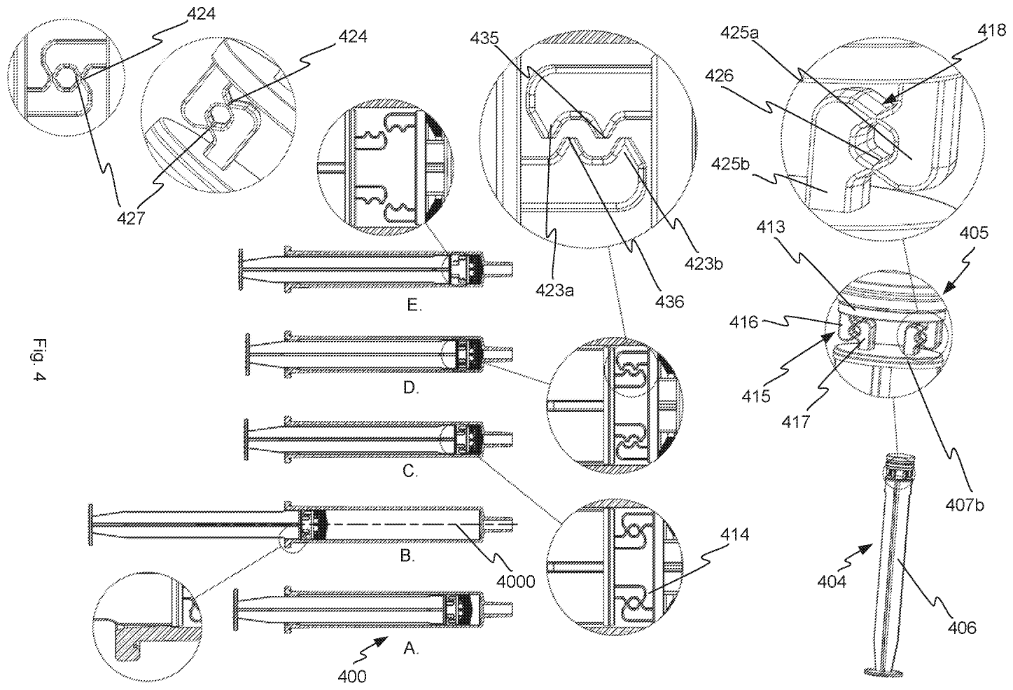

FIG. 3 shows a plunger rod 304 according to a third embodiment. The plunger rod 304 has a distal end top 305 and an elongated proximal shaft 306. The releasable connection 315 is similar to the embodiment shown in FIG. 2. The releasable connection 315 comprises the distal top rupture means 316, having two distal top ribs 325a eccentrically located on the proximal bottom plate 313. Two proximal shaft ribs 325b of the proximal shaft rupture means 317 are eccentrically arranged on the distal end top 307b. The proximal shaft ribs 325b are arranged laterally relative the distal top ribs 325a. The distal top ribs 325a and the proximal shaft ribs 325b extend along the axis 3000.

The distal top ribs 325a and the proximal shaft ribs 325b are disposed linearly along a central diameter of the bottom surface 313 and the distal end plate 307b respectively. The proximal shaft ribs 325b are positioned centrally, closer to the axis 3000, in relation to the distal top ribs 325a.

The distal top ribs 325a each comprises a distal rib protrusion 323a and the proximal shaft ribs 325b each comprises a proximal rib protrusion 323b. The distal top ribs 325a and the proximal shaft ribs 325b are connected to each other through the distal rib protrusions 323a and the proximal shaft protrusions 323b. The distal rib protrusions 323a and the proximal shaft protrusions 323b extend radially from the distal top ribs 325a and the proximal shaft ribs 325b.

The distal rib protrusions 323a on the ribs 325a points centrally. The proximal shaft protrusions 323b points laterally, along the same diameter on which the distal top ribs 325a and proximal shaft ribs 325b are linearly arranged. Each distal rib protrusion 323a is connected to a corresponding proximal rib protrusion 323b. The corresponding distal rib protrusions 323a and proximal rib protrusion 323b are arranged in longitudinal level with each other such that they face each other. The connected distal rib protrusion 323a and proximal rib protrusion 323b form an hour glass shaped connection between the distal top ribs 325a and the proximal shaft ribs 325b.

The hour glass shaped connections formed by the distal rib protrusion 323a and proximal rib protrusion 323b may comprise a weakening groove 324 (shown in enlarged view of Position D in FIG. 3). The groove 224 is positioned at the node of the hour glass shaped connection. The groove 324 facilitates a sharp rupture between the cones 323.

The distal top ribs 325a and the proximal shaft ribs 325b are hexagonal and non-symmetrical. An advantage with this embodiment is that it is easy to optimise the behaviour of the compression failure.

The use positions A-D are shown in FIG. 3. In position C, enlarged views show the third embodiment with and without the optional groove 324. In position D, the proximal shaft rupture means 317 has been released from the distal top rupture means 316 and the proximal shaft ribs 325b have been pushed into the cavity 314. Once compression failure has been established (position D) the plunger rod 304 may be retracted from the barrel 302 (position E—not shown in FIG. 3). When rupture has occurred, the distal rib 325a has a distal rib fracture surface 335 and the proximal shaft rib 325b has a proximal shaft rib facture surface 336. The distal rib fracture surface 335 points centrally. The proximal shaft rib facture surface 336 points laterally.

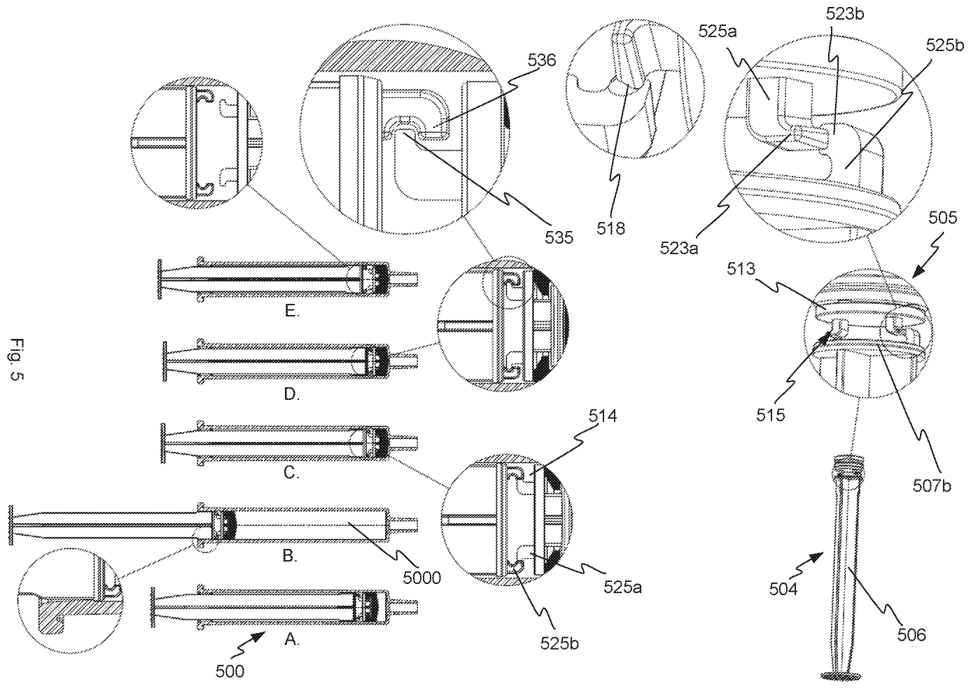

A fourth embodiment of a plunger rod 404 is shown in FIG. 4. The plunger rod 404 has a distal plunger top 405 and an elongated proximal shaft 406. The releasable connection 415 is similar to the embodiment shown in FIG. 2, and comprises rupture means 416 having two distal top ribs 425a eccentrically located on the proximal bottom plate 413 of the distal end top 405. Two proximal shaft ribs 425b of the proximal shaft rupture means 417 are eccentrically arranged on the distal end top 407b.

The distal top ribs 425a and the proximal shaft ribs 425b extend along the axis 4000. Each distal top rib 425a comprises two distal rib protrusions 423a. Each proximal shaft rib 425b comprises two proximal rib protrusions 423b. The distal rib protrusions 423a and proximal rib protrusions 423b extend substantially perpendicular from the distal top ribs 425a and the proximal shaft ribs 425b and the axis 4000.

The distal top ribs 425a and the proximal shaft ribs 425b are connected to each other through the distal rib protrusions 423a and the proximal shaft protrusions 423b. The distal rib protrusions 423a and the proximal shaft protrusions 423b extend radially from the distal top ribs 425a and the proximal shaft ribs 425b.

The distal rib protrusions 423a on the ribs 425a points centrally. The proximal shaft protrusions 423b points laterally, along the same diameter on which the distal top ribs 425a and proximal shaft ribs 425b are linearly arranged. Each distal rib protrusion 423a is connected to a corresponding proximal rib protrusion 423b, at a contact area 418. The corresponding distal rib protrusions 423a and proximal rib protrusion 423b are arranged in longitudinal level with each other such that they face each other. The connected distal rib protrusion 423a and proximal rib protrusion 423b form an hour glass shaped connection between the distal top ribs 425a and the proximal shaft ribs 425b.

When rupture has occurred, the distal ribs 425a have a distal rib fracture surface 435 and the proximal shaft ribs 425b have a proximal shaft rib facture surface 436. The distal rib fracture surface 435 points centrally. The proximal shaft rib facture surface 436 points laterally.

The distal top ribs 425a and the proximal shaft ribs 425b are disposed linearly along a central diameter of the bottom surface 413 and the distal end plate 407 respectively. The proximal shaft ribs 425b are positioned closer to the axis 4000 than the distal top ribs 425a.

In this embodiment, the protrusions 423 are shaped like triangular prisms. This results in that the contact area 418 has an elongated width 426. This embodiment of the releasable connection 415 has good stability due to the elongate width 426. The contact area 418 may further comprise a reinforcement 427 facing the elongate shaft 406. The contact area 418 may further comprise a groove 424 facing the distal end top 405.

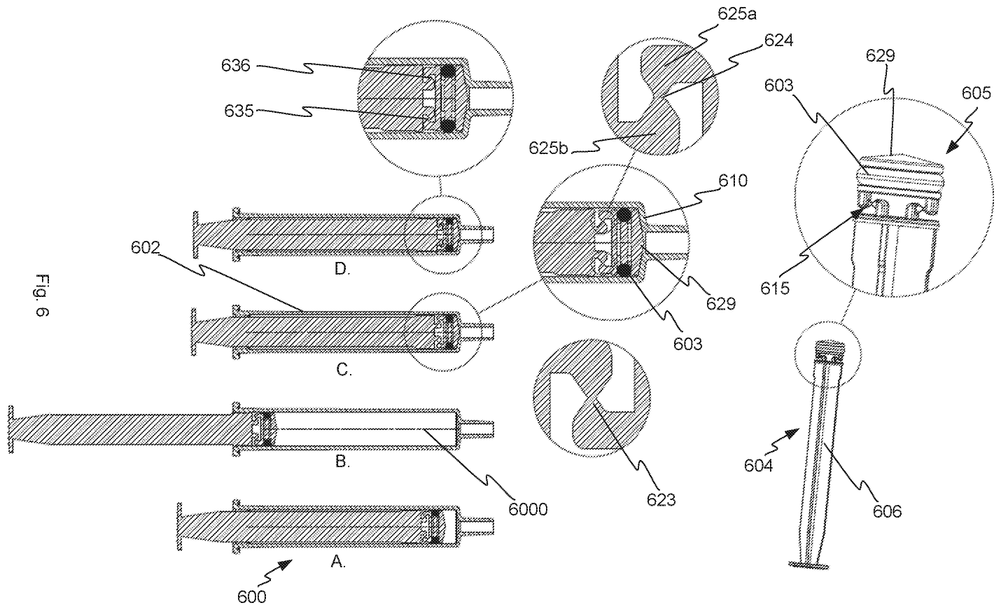

FIG. 5 shows a plunger rod 504 according to a fifth embodiment. The distal end plate 507b and the proximal bottom plate 513 each comprises two eccentrically disposed distal top ribs 525a and proximal shaft ribs 525b.

Each distal top rib 525a comprises a distal rib protrusions 523a. Each proximal shaft rib 525b comprises a proximal rib protrusion 523b. The distal top ribs 525a and the proximal shaft ribs 525b are connected to each other through the distal rib protrusions 523a and the proximal shaft protrusions 523b. The distal rib protrusions 523a and the proximal shaft protrusions 523b extend radially from the distal top ribs 525a and the proximal shaft ribs 525b.

The distal rib protrusions 523a on the ribs 525a points centrally. The proximal shaft protrusions 523b points laterally. Each distal rib protrusion 523a is connected to a corresponding proximal rib protrusion 523b, at a contact area 518. The corresponding distal rib protrusions 523a and proximal rib protrusion 523b are arranged in longitudinal level with each other such that they face each other.

As opposed to the embodiments shown in FIGS. 1-4, the distal rib protrusions 523a and the proximal shaft protrusions 523b in this embodiment are not facing each other linearly. Instead, the contact area 518 is formed through an overlap between side edges of the distal rib protrusions 523a and the proximal shaft protrusions 523b. Each centrally facing side edge of the distal rib protrustions 523a are connected to a centrally facing side edge of the proximal rib protrusion 523b.

The two distal top ribs 525a are eccentrically arranged in relation to the axis 5000, and relative the proximal shaft ribs 525b. As shown in the enlarged view of Position C, the distal top rib 525a is arranged centrally relative the proximal shaft rib 525b. Further, as can be seen in the top enlarged view of the plunger rod 504, the distal top rib 525a is also displaced horizontally relative the proximal shaft rib 525b, the horizontal direction being perpendicular to the longitudinal axis 5000. Hence, the distal top ribs 525a and the proximal shaft ribs 525b are not arranged linearly along a diameter of the bottom surface 513 and the distal end plate 507b.

Preferably, the contact area 518 is small, such as 0.2 mm². The overlap in the contact area 518 provides stability during rotations of the plunger rod 504 in the barrel 502.

When the plunger rod 504 is pushed into the front position and excess load is applied, the distal rib protrusions 523a and proximal rib protrusions 523b disengage from each other and the proximal shaft ribs 525b are pushed into the cavity 514, as shown in the enlarged view of position D in FIG. 5.

When rupture has occurred, the distal ribs 525a have a distal rib fracture surface 535 and the proximal shaft ribs 525b have a proximal shaft rib facture surface 536. The distal rib fracture surfaces 535 and the proximal shaft rib facture surfaces 536 face the centre axis 5000.

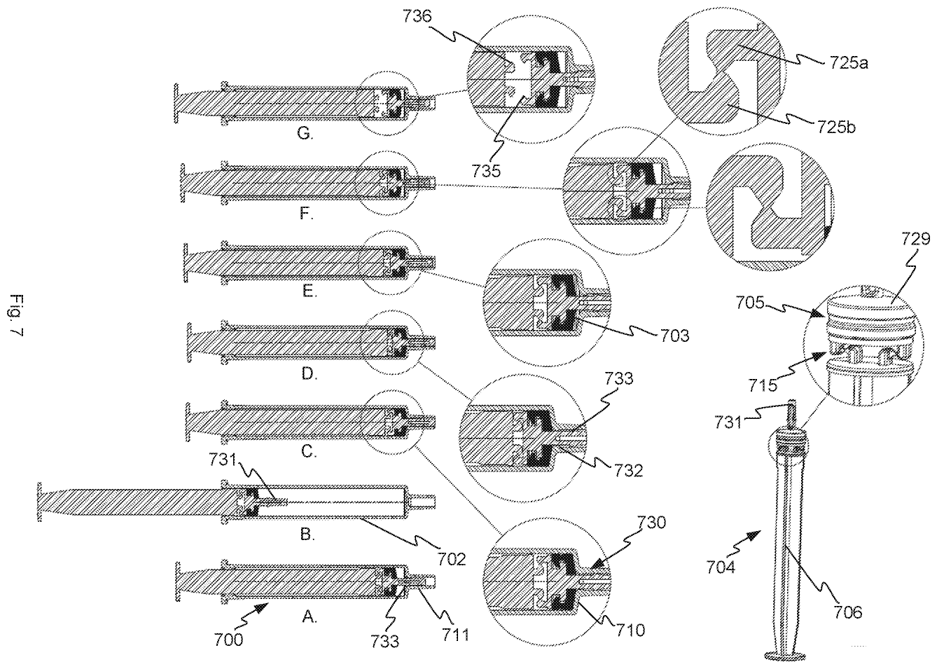

FIG. 6 shows a plunger rod 604 comprising the releasable connection 615 according to embodiment number three (FIG. 3), wherein the washer is an O-ring seal 603. Since the distal plunger top 605 is not covered by the washer as shown in FIG. 3, in this embodiment, the compression failure is performed having two solid and rigid polymers, such as polypropylene (PP), in direct contact with each other. The distal end 610 of the barrel 602 is in direct contact with a top surface 629 of the distal plunger top 605 in position C and D. This yields a sharper and more distinct rupture of the rupture cones 623. The O-ring seal 603 is fabricated in an attenuating material, such as an elastomer, for instance synthetic rubber or silicone. The O-ring seal 603 may be combined with any other of the embodiments disclosed herein.

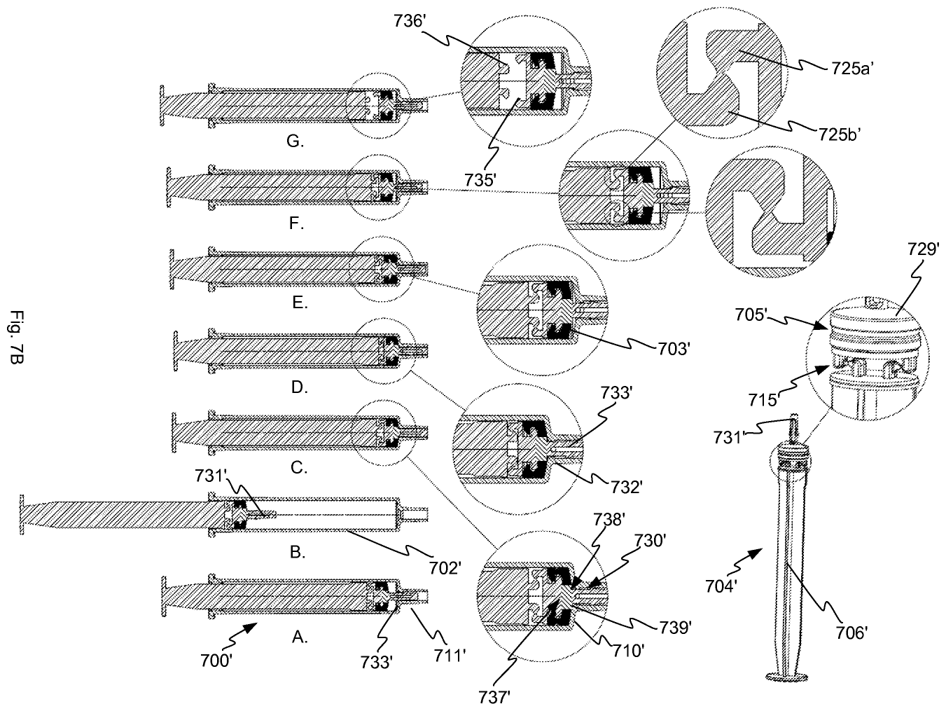

FIG. 7 shows a plunger rod 704 according to embodiment number three wherein the syringe 700 further comprises an additional locking mechanism 730. The plunger top 705 comprises an end protrusion 731 extending through the washer 703 from the top surface 729. The distal tip 711 of the syringe 700 comprises an internal retaining wall 732 configured to engage a retaining portion 733 disposed on the end protrusion 731. The releasable connection 715 is of the same type as shown in FIG. 3 and FIG. 6.

The addition of the locking mechanism 730 generates an auto-disabled syringe 700 comprising features enabling both compression failure and tension failure. As seen in Position A in FIG. 7, the retaining portion 733 is located proximally of the distal end 710 of the barrel 702 and the distal tip 711 of the syringe 700. When the retaining protrusion 733 on the end protrusion 731 is pushed passed the retaining wall 732, the locking mechanism is activated. This is shown in the enlarged view of Position C. In Position D, both the locking mechanism 730 has been engaged and the releasable connection 715 has been released. In Position E, the locking mechanism 730 is activated, but the releasable connection 715 is still intact. If the releasable connection 715 fails to rupture during compression, the retaining portion 733 will engage in the retaining wall 732 as shown in the enlarged view of Positon F, and the plunger top 705 will be released from the plunger rod 704, as shown in Position G.

The additional locking mechanism 730 may be combined with any other releasable connection 115, 215, 315, 415, 515, 615, 815 of the embodiments disclosed herein.

In FIG. 7B, a further embodiment of the syringe 700 of FIG. 7 is shown. The syringe 700′ has a plunger rod 704′ and an additional locking mechanism 730′ of the same type as shown in FIG. 7. The distal plunger top 705′ comprises an end protrusion 731′ extending through the washer 703′ from the top surface 729′. The distal tip 711′ of the syringe 700′ comprises an internal retaining wall 732′ configured to engage a retaining portion 733′ disposed on the end protrusion 731′. The releasable connection 715′ is of the same type as shown in FIGS. 3, 6 and 7.

In addition the washer 703′ has a centre aperture 737′. The distal plunger top 705′ has an elevated socket 738′ extending through said centre aperture 737′ of the washer 703′. The centre aperture 737′ may for instance be formed by injection moulding or by punching. A diameter of the socket 738′ is in the same range or slightly broader than a diameter of the centre aperture 737′. If the diameter of the socket 738′ is larger than the width of the centre aperture 737′, the socket 737′ may be fitted into the aperture 738′ by force fitting.

A distal end surface 739′ of the socket 738′ lies in the same longitudinal level as the washer 703′, as can be seen for instance in the enlarged portion of for instance Position C-G in FIG. 7B. When the distal plunger top 705′ reaches the distal end 710′, the distal end surface 739′ of the socket 738′ and the distal end 710′ of the barrel 702′ come into direct contact with each other. Since the socket 738′ and the distal end 710′ preferably comprise two solid and rigid polymers, such as polypropylene (PP), a sharper and more distinct rupture between the plunger rod 704′ and the distal plunger top 705′, in the same way as described for the embodiment shown in FIG. 6.

Similarly to the embodiment shown in FIG. 3, when rupture has occurred between the distal plunger top 605, 705, 705′ and the plunger rod 604, 704, 704′ in FIGS. 6, 7 and 7B, the distal rib 625a, 725a, 725a′ has a distal rib fracture surface 635, 735, 735′ and the proximal shaft rib 625b, 725b, 725b′ has a proximal shaft rib facture surface 636, 736, 736′. The distal rib fracture surface 635, 735, 735′ points centrally. The proximal shaft rib facture surface 636, 736, 736′ points laterally.

With reference to FIG. 8, a plunger rod 804 according to an eighth embodiment is shown. The plunger rod 804 is designed to be arranged in a pre-filled syringe 800, and comprises an elongated proximal shaft 806 and a distal end top 805. The distal end top 805 comprises a proximal bottom plate 813 connected to a distal end plate 807 through a releasable connection 815, which is similar to the fifth embodiment number fifth in FIG. 5. The barrel 802 is preferably made of glass. In Position A, the lumen 808 of the barrel 802 is filled with a liquid or drug. The syringe 800 comprises a needle 840 covered with a needle cap 841. The distal plunger top 805 comprises a threaded protrusion 845, extending from the distal surface of the bottom plate 813.

When mounting pre-filled syringes 800, preferably, the washer 803 is first inserted into the aperture 809 of the barrel 802. Subsequently, the plunger rod 804 is inserted into the aperture 809 by threading the threaded protrusion 845 into the washer 803. The releasable connection 815 in this embodiment is adapted to withstand such threading during mounting without being damaged.

The releasable connection 815 comprises distal top ribs 825a and proximal shaft ribs 825b eccentrically arranged on the bottom surface 813 and the distal end plate 807, respectively. The bottom surface 813 and the distal end plate 807 each comprise two distal top ribs 825a, and two proximal shaft ribs 825b respectively. The proximal shaft ribs 825b are arranged linearly along a diameter of the distal end plate 807b. The distal top ribs 825a are arranged centrally, closer to the axis 8000, relative the proximal shaft ribs 825b. The distal top ribs 825a each comprise a distal rib protrusion 823a. The proximal shaft ribs 825b each comprise a proximal rib protrusion 823b. The distal rib protrusions 823a and the proximal rib protrusions 823b are connected to each other at contact areas 818. The contact areas 818 are located in a bonding plane extending along and through the axis 8000. The two distal top ribs 825a are arranged on opposite sides relative the bonding plane. The two proximal shaft ribs 825b are arranged on opposite sides relative the bonding plane. The two formed contact areas 818 are central lateral overlaps between a distal rib protrusion 823a and a proximal rib protrusion 823b.

Hence, the construction of the releasable connection 815 in FIG. 8 is an overlapping and crosswise connection. The central lateral overlaps of the contact areas 818 enable the releasable connection 815 to withstand rotation, for instance when assembling and mounting a pre-filled syringe. When the plunger rod 804 is rotated in one direction, the distal rib protrusions 823a will be pushed against the proximal rib protrusions 823b at the contact area 818. If the plunger rod 804 is rotated in the opposite direction, the proximal rib protrusions 823b will be pushed against the distal rib protrusions 823a at the contact area 818. Thus, the construction of the releasable connection 815 is durable during both clockwise and counter clockwise rotations. Preferably, the contact area 818 is minimal, such as 0.2 mm².

The releasable connection 815′ comprise an eccentrically arranged distal top rib 825a and a proximal shaft rib 825b. The distal top rib 825a is disposed on the bottom surface 813 and the proximal shaft rib 825b is arranged on the distal end plate 807b. In the same way as described for the releasable connection 815, the distal top rib 825a and the proximal shaft rib 825b are connected to each other in an overlapping manner. The releasable connection 815′ is for instance suitable for syringes having a small diameter of the barrel 802. In the same way as described for the releasable connection 815, the overlap between the distal top rib 825a and the proximal shaft rib 825b cause the releasable connection 815′ to withstand rotations of the plunger rod 804.

When rupture has occurred between the distal plunger top 805 and the plunger rod 804 in FIG. 8, the distal rib 825a has a distal rib fracture surface 835 and the proximal shaft rib 825b has a proximal shaft rib facture surface 836.

The embodiments shown in FIGS. 1-8 minimizes the production costs of auto-disabled syringes. Since the constructions rely on rupture due to compression failure and do not depend on interactions between the plunger rod and the syringe barrel, the plunger rods herein can be used together with already existing syringes (embodiment 1-6) or already existing pre-filled syringes (embodiment 8). The seventh embodiment require the formation of the internal retaining recess 732. However, such feature is easily manufactured during injection moulding. The designs of the disclosure herein are more simple than current syringes provided with features for tension failure or spring loaded syringes. Hence, the productions costs can be lowered to the same level or lower as for conventional syringes or conventional pre-filled syringes.

In addition, the new designs provide good stability enabling high speed mounting processes. Further, the plunger rods disclosed herein may be manufactured of different polymers suitable for medical devices, having altering materialistic properties. The brittleness and impact strength of the polymer will affect the behaviour of the plunger rod. Thus, the possibility of optimisation of the strength during extraction of the plunger rod to fill the barrel with a drug and of the compression failure is wide.

All embodiments herein may comprise fewer or more ribs than described for each example. Further, each ribs may comprise fewer or more protrusions extending from said ribs. The ribs describes as situated closer to an axis may be arranged on either the distal end plate of the proximal elongate shaft or the proximal bottom surface of the distal plunger top. The ribs may have various dimensions, i.e. different lengths and widths. The protrusions may have altering shapes and dimensions.

Further, the invention has mainly been described with reference to a few embodiments. However, as is readily understood by a person skilled in the art, other embodiments than the ones disclosed above are equally possible within the scope of the invention, as defined by the appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A plunger rod for use in connection with a syringe barrel, the plunger rod comprising:
a distal plunger top, having a proximal bottom surface; and
a proximal elongated shaft having a proximal end adapted to receive an applied force along an axis, and a distal end plate adapted to transmit the applied force along the axis;
wherein the plunger rod is adapted to be inserted into a barrel of a syringe extending along the axis; and
wherein the distal plunger top and the proximal elongated shaft are connected to each other by a releasable connection, the releasable connection having
distal top rupture means including at least one distal top rib disposed eccentrically from the axis on the proximal bottom surface, and
proximal shaft rupture means including at least one proximal shaft rib disposed eccentrically from the axis on the distal end plate;
wherein each of the at least one distal top rib is connected to a corresponding at least one proximal shaft rib, the top and the proximal shaft ribs being connected at a contact area; and
wherein the distal top rupture means is for detaching from the proximal shaft rupture means due to compression failure when the distal plunger top is pushed into a distal end of the barrel.

2. The plunger rod according to claim 1, wherein the distal top and the proximal shaft ribs extend along the axis from the distal end plate and from the proximal bottom surface, respectively.

3. The plunger rod according to claim 1, wherein the plunger rod further comprises a cavity, formed between the proximal shaft ribs of the distal end plate and the proximal bottom surface, configured to receive the proximal shaft ribs when the force is transmitted along the axis to the distal plunger top and is causing the distal top ribs and the proximal shaft ribs to be released from each other.

4. The plunger rod according to claim 1, wherein the distal top ribs are radially displaced relative to the proximal shaft ribs.

5. The plunger rod according to claim 1, wherein when the distal top rib and the proximal shaft rib are disconnected from each other, the distal top rib has a distal rib fracture surface and the proximal shaft rib has a proximal shaft rib fracture surface pointing centrally and laterally, respectively, relative to the axis.

6. The plunger rod according to claim 1, wherein the distal top ribs and the proximal shaft ribs are aligned linearly along a diameter of the proximal bottom surface and the distal end plate.

7. The plunger rod according to claim 1, wherein the distal top and the proximal shaft ribs extend along the axis; and
wherein a centrally facing side of each distal top rib is connected to a laterally facing side of a corresponding proximal shaft rib or vice versa.

8. The plunger rod according to claim 7, wherein the distal top ribs are angled away from the axis and the proximal shaft ribs are angled towards the axis or vice versa.

9. The plunger rod according to claim 1, wherein the distal top and the proximal shaft ribs are arranged such that a side edge of the distal top rib, perpendicular to a side of the distal top rib facing away from the axis, is connected to a side edge of a proximal shaft rib, perpendicular to a side of the proximal shaft rib facing away from the axis.

10. The plunger rod according to claim 1, wherein the distal top ribs and the proximal shaft ribs are in the shape of substantially a pyramid, a cube, a cuboid, a prism, a cone, a tetrahedron, a cylinder or any other geometrical three dimensional shape enabling the connection between the distal top ribs and the proximal shaft ribs.

11. The plunger rod according to claim 1, wherein the distal top rib and the proximal shaft rib comprise at least one distal rib protrusion and a proximal rib protrusion, respectively.

12. The plunger rod according to claim 11, wherein each distal rib protrusion faces and is arranged opposite to a corresponding proximal rib protrusion; and
wherein the distal rib and the proximal rib protrusions form the connection between the distal top ribs and the proximal shaft ribs.

13. The plunger rod according to claim 12, wherein the connection formed between each distal rib protrusion and each corresponding proximal rib protrusion have a longitudinal cross-section along the axis with an hour-glass like shape.

14. The plunger rod according to claim 1, wherein the distal plunger top comprises a washer; and
wherein the washer is adapted to be in fluid-tight engagement with a tubular wall of the barrel, the washer being displaceable along the axis within the barrel.

15. The plunger rod according to claim 14, wherein the washer is an O-ring seal.

16. The plunger rod according to claim 1, wherein the distal plunger top further comprises an end protrusion distally arranged on the distal plunger top and including a retaining portion configured to engage an internal retaining recess of the end protrusion.

17. The plunger rod according to claim 16, wherein the distal plunger top comprises an elevated socket having a distal end surface arranged at a proximal end of the end protrusion, such that the distal end surface is brought into direct contact with a distal end of the barrel when the distal plunger top is pushed into the distal end.

18. The plunger rod according to claim 11, wherein the distal rib and the proximal rile protrusions are in the shape of substantially a pyramid, a cube, a cuboid, a prism, a cone, a tetrahedron, or any other geometrical three dimensional shape enabling the connection between the distal rib protrusions and the proximal rib protrusions.

19. The plunger rod according claim 1, wherein the contact area comprises a weakening, arranged on a distally facing side of the contact area.

20. The plunger rod according to claim 1, wherein the contact area comprises a reinforcement, arranged on a proximally facing side of the contact area.

21. The plunger rod according to claim 1, wherein the proximal bottom surface has a diameter smaller than a diameter of a circumferential flange formed on an inner wall of the barrel.

22. A syringe comprising:
a barrel having a syringe tip arranged at a distal end of the barrel the barrel being adapted to contain a drug, the barrel having a tubular wall extending proximally from the distal end, whereby the barrel extends along an axis;
wherein the barrel includes an aperture arranged at a proximal end of the barrel and;
wherein the syringe further comprises a plunger rod according to claim 1 extending along the axis.

23. The syringe according to claim 22, wherein the syringe contains the drug.

24. The syringe according to claim 22, wherein the barrel is made of plastic or glass.

25. The syringe according to claim 22, wherein the syringe tip comprises a needle.
